(19) United States
(12) Patent Application Publication
(10) Pub. No.: US 2021/0315656 A1
(43) Pub. Date: Oct. 14, 2021

(54) TORQUE LIMITING HANDLE FOR MEDICAL INSTRUMENT

(71) Applicant: Nextremity Solutions, Inc., Warsaw, IN (US)

(72) Inventors: Gregory Denham, Warsaw, IN (US); Joseph Whitley, Leesburg, IN (US); Ryan Schlotterback, Fort Wayne, IN (US)

(73) Assignee: Nextremity Solutions, Inc., Warsaw, IN (US)

(21) Appl. No.: 17/210,643

(22) Filed: Mar. 24, 2021

Related U.S. Application Data
(60) Provisional application No. 63/007,998, filed on Apr. 10, 2020.

(51) Int. Cl. A61B 90/00; A61B 17/00

(52) U.S. Cl. CPC A61B 90/03; A61B 17/00; A61B 2017/00398; A61B 2017/00464; A61B 2090/031

(57) ABSTRACT

A torque limiting handle for a medical instrument is provided that includes a tool connector, a torque coupler, and a rear power housing. The handle limits the amount of torque in one direction while allowing for maximal torque in the opposite direction. In one aspect, the torque limiting handle may include a drive shaft removeably connected to a power tool or, alternatively, to a handle grip for manual manipulation.

### BACKGROUND OF THE INVENTION

### Technical Field

The present invention generally relates to handles for a surgical instrument or device, and more particularly, to quick disconnect handles for a surgical instrument or device that limits the amount of torque in one direction while allowing for maximal torque in the opposite direction.

### Background Information

Current disposable handles on the market tend to be very expensive. Many of the handles on the market are configured for manual manipulation for the purpose of limiting torque while, for example, seating a screw in bone to prevent damage to the screw or driver or bone. Too low of a torque, however, may correspond to low initial mechanical implant stability and too high of a torque may lead to damage to the screw or driver, or even to avascular bone necrosis.

Thus, a need exists for a handle that can limit the amount of torque that can be applied to a drive shaft of a surgical instrument or device.

### SUMMARY OF THE INVENTION

Briefly, the handle constructed in accordance with one or more aspects of the present invention satisfies the need for limiting the amount of torque, in one direction that can be applied to a drive shaft of a surgical instrument or device by use of either manual or power application of torsion.

In one aspect of the present invention, an apparatus for releasably holding a surgical tool is provided. The apparatus comprises a tool connector, a torque coupler, and a rear power housing. The tool connector includes a longitudinal axis, a proximal end and a distal end. The tool connector further includes a tool engagement body and a mounting post extending longitudinally along the longitudinal axis from the tool engagement body to the distal end. The tool engagement body includes a tool engagement opening at the proximal end communicating with a longitudinal bore extending through at least a portion of the tool engagement body along the longitudinal axis. The longitudinal bore configured to releasably coupled the surgical tool.

The torque coupler incudes a cylindrical body defining a through hole and an outer surface. The mounting post of the tool connector passes through the through hole of the cylindrical body. The torque coupler further includes a plurality of fingers extending radially outward from the outer surface.

The rear power housing is rotatably coupled to the mounting post of the tool connector at the distal end. The rear power housing includes a longitudinal axis, a body and a drive shaft extending longitudinally along the longitudinal axis from the body of the rear power housing. The body includes a cavity defining an inner surface and a plurality of teeth projecting radially inward from the inner surface.

During rotation of the rear power housing in a first direction, the plurality of fingers slidably engage the plurality of teeth to limit the applied torque of the torque coupler and the tool connector from the rear power housing. During rotation of the rear power housing in a second direction, the plurality of teeth prevent movement of the plurality of fingers to allow maximal applied torque of the torque coupler and the tool connector from the rear power housing.

In another aspect, the rear power housing of the apparatus for releasably holding a surgical tool is removeably attachable to a handle grip.

These, and other objects, features and advantages of this invention will become apparent from the following detailed description of the various aspects of the invention taken in conjunction with the accompanying drawings.

### BRIEF DESCRIPTION OF THE DRAWINGS

The present invention will be understood more fully from the detailed description given hereinafter and from the accompanying drawings of the certain embodiments of the present invention, which, however, should not be taken to limit the invention, but are for explanation, illustration and understanding only.

FIG. 1A depicts an exploded perspective view of a handle with an optional handle grip constructed in accordance with one or more aspects of the present invention;

FIG. 1B depicts a cross-sectional view of the exploded perspective view shown in FIG. 1 rotated counterclockwise ninety degrees and taken along the plane 1-1;

FIG. 2A depicts a perspective view of a tool connector for a handle constructed in accordance with one or more aspects of the present invention;

FIG. 2B depicts a cross-sectional view of the tool connector shown in FIG. 2A rotated clockwise ninety degrees and taken along the plane 2-2;

FIG. 2C depicts a partial cross-sectional view illustrating one example of a coupling mechanism attaching a distal end of a tool connector to a rear power housing constructed in accordance with one or more aspects of the present invention;

FIG. 3A depicts a perspective view of a torque coupler for a handle constructed in accordance with one or more aspects of the present invention;

FIG. 3B depicts a cross-sectional view of the torque coupler shown in FIG. 3A taken along the plane 3-3;

FIG. 4A depicts a perspective view of a rear power housing for a handle constructed in accordance with one or more aspects of the present invention;

FIG. 4B depicts a cross-sectional view of the rear power housing shown in FIG. 4A taken along the plane 4-4;

FIG. 5A depicts a perspective view of one example of an optional handle grip for a handle constructed in accordance with one or more aspects of the present invention;

FIG. 5B depicts a cross-sectional view of the handle grip shown in FIG. 5A rotated counterclockwise ninety degrees and taken along the plane 5-5;

FIG. 6A depicts a perspective view of an alternative embodiment of a handle grip for a handle constructed in accordance with one or more aspects of the present invention;

FIG. 6B depicts a perspective view of an alternative embodiment of a handle grip for a handle constructed in accordance with one or more aspects of the present invention;

FIG. 6C depicts a perspective view of an alternative embodiment of a handle grip for a handle constructed in accordance with one or more aspects of the present invention.

FIG. 7A depicts a side view of one embodiment of an assembled handle with one example of an optional handle grip constructed in accordance with one or more aspects of the present invention;

FIG. 7B depicts a cross-sectional view of the assembled handle shown in FIG. 7A rotated counterclockwise ninety degrees and taken along the plane 7-7;

FIG. 8A depicts a side view of an assembled handle constructed in accordance with one or more aspects of the present invention for use with either an optional handle grip or a power tool;

FIG. 8B depicts a cross-sectional perspective view of the assembled handle shown in FIG. 8A taken along the plane 8-8; and

FIG. 8C depicts a cross-sectional view of the assembled handle shown in FIG. 8A taken along the plane 8-8.

### DETAILED DESCRIPTION OF THE INVENTION

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without some of these specific details. In other instances, well-known structures are not shown in detail in order to avoid unnecessary obscuring of the present invention.

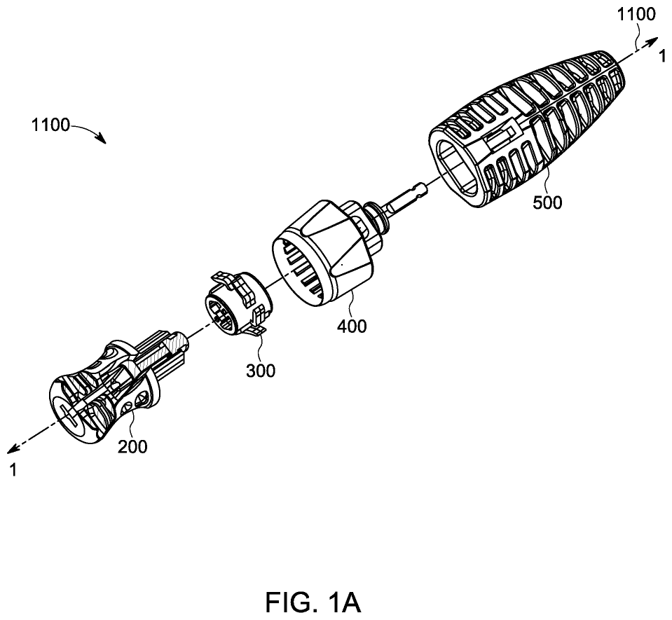

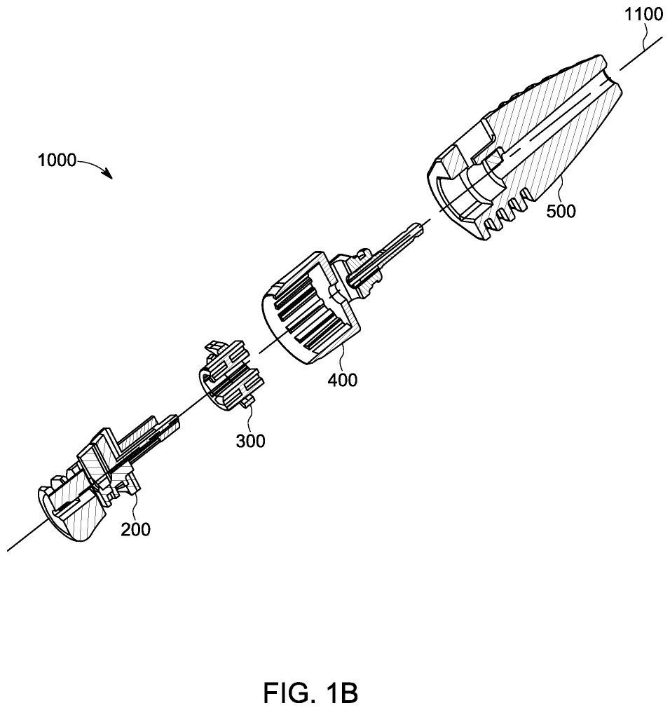

Thus, all implementations described below are exemplary implementations provided to enable persons skilled in the art to make or use the embodiments of the disclosure and are not intended to limit the scope of the disclosure, which is defined by the claims. As used herein, the word “exemplary” or “illustrative” or “example”, and derivatives thereof, means “serving as an example, instance, or illustration.” Any implementation described herein as “exemplary” or “illustrative” or “example”, and derivatives thereof, is not necessarily and should not be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1A.

Furthermore, there is no intention to be bound by any expressed or implied theory presented in the preceding technical field, background, brief summary or the following detailed description. It is also understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise. While this invention is satisfied by embodiments in many different forms, there is shown in the drawings, and will herein be described in detail, one or more embodiments of the present invention with the understanding that the present disclosure is to be considered as exemplary of the principles and aspects of the invention and is not intended to limit the invention to the embodiments illustrated. The scope of the invention will be pointed out in the appended claims.

In short, a handle constructed in accordance with one or more aspects of the present invention provides a quick disconnect handle for use with a surgical instrument, such as, for example, a drill bit or screwdriver, that limits the amount of torque in one direction while allowing for maximal torque in the opposite direction. The handle may be operated manually by an optional removable handle grip or by power with a power instrument removably attached.

Referring now to FIGS. 1A and 1B, there is shown an exploded perspective view of a handle 1000 constructed in accordance with one or more aspect of the present invention. As illustrated in FIGS. 1A and 1B, handle 1000 may include a tool connector 200, a torque coupler 300, a rear power housing 400 and an optional handle grip 500. Each of tool connector 200, a torque coupler 300, a rear power housing 400 and an optional handle grip 500 share a common longitudinal or rotational axis 1100. Handle is configured to couple to a medical instrument or tool such as, for example, a screw driver or drill. In one example, a screw driver or drill is positively and removably grasped or coupled to a coupling mechanism provided at a proximal end 212 of tool connector 200 of handle 1000. The coupling mechanism, one example of which will be described in more detail below, is configured to transfer the torque applied to handle 1000 to the medical instrument or tool (not shown) removably attached to tool connector 200.

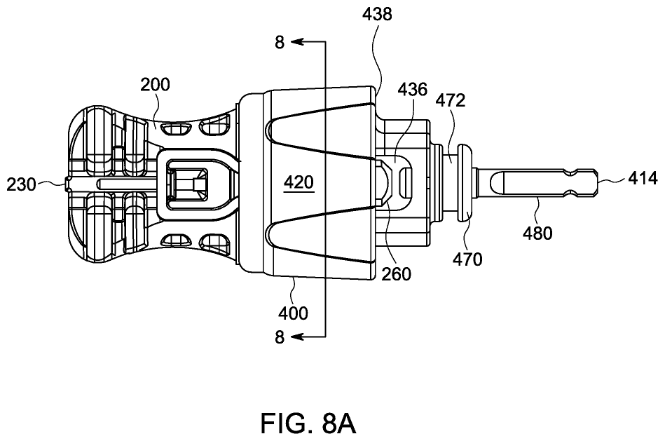

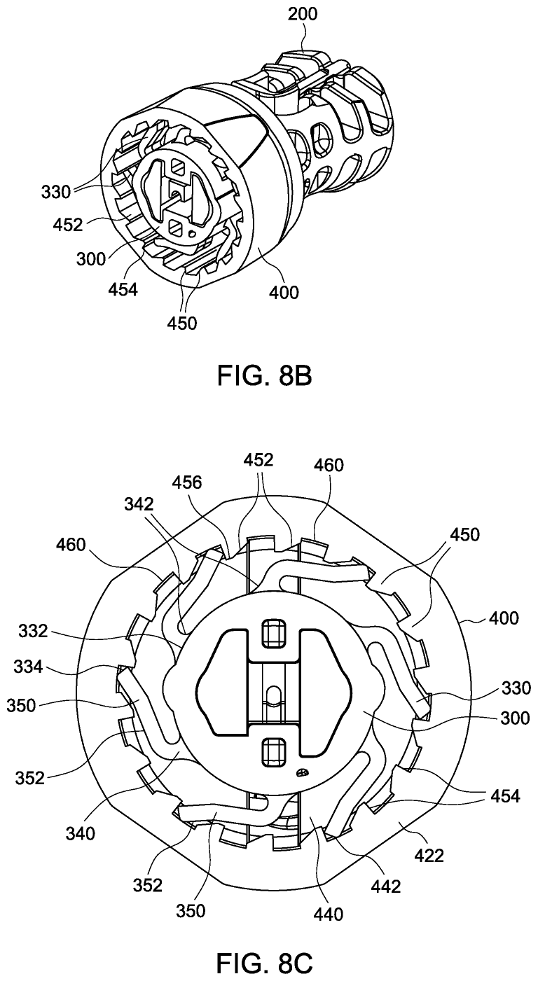

Tool connector 200, torque coupler 300 and rear power housing 400 are assembled and not separable during use. An example of this assembly is illustrated in FIG. 8A. The assembly of tool connector 200, torque coupler 300 and rear power housing 400 may readily and removably couple or attach, to drive shank 480 at distal end 414 of rear power housing 400, to a power instrument or tool, such as, for example, a cordless power drill. Instead of attaching to a power instrument or tool, the assembly of tool connector 200, torque coupler 300 and rear power housing 400 may also readily and removably couple or attach to an optional handle grip 500 for manual operation such as, for example, manually inserting a screw. Handle grip 500 may be removed or added by an end user to transition between power and manual application of torsion applied to handle 1000.

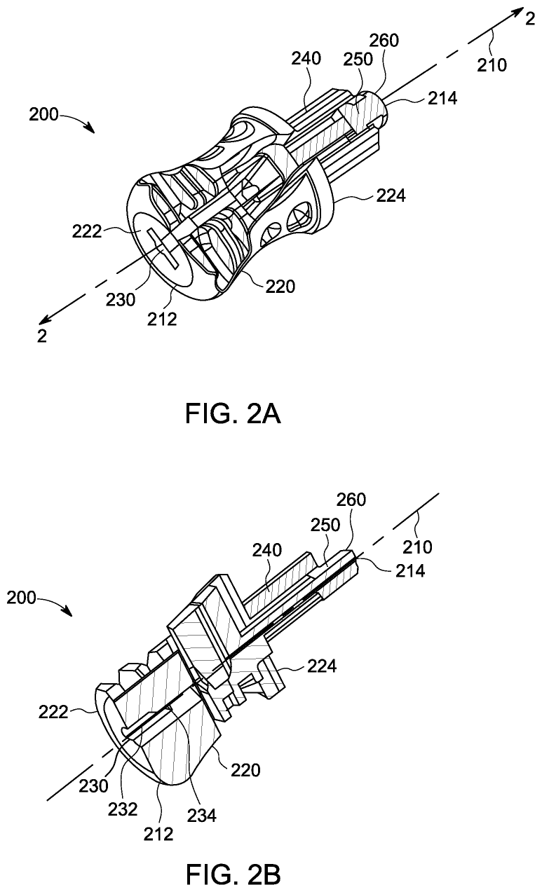

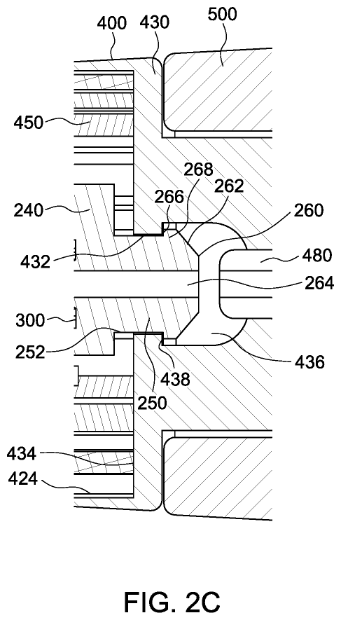

FIGS. 2A and 2B illustrate one example of a tool connector 200 constructed in accordance with one or more aspects of the present invention. As shown in FIG. 2A, tool connector 200 includes a longitudinal axis 210, a proximal end 212 and a distal end 214. During assembly and operation, longitudinal axis 210 aligns with longitudinal axis 1100 of handle 1000. Tool connector 200 also includes a tool or instrument engagement body 220 extending from proximal end 212 and a mounting post 240 extending longitudinally from tool or instrument engagement body 220 to distal end 214.

Tool or instrument engagement body 220 includes a first side 222 at proximal end 212 and a second side 224 from which mounting post 240 extends therefrom. Tool or instrument engagement body 220 may include a longitudinal bore 232 in communication with tool engagement opening 230 disposed through at least a portion of body 220 along longitudinal axis 210 and shaped to receive and removably couple or retain a drive shank of a surgical tool or instrument, such as, for example, a drill or a screwdriver.

There are many coupling mechanisms known in the art that removably couple or retain a surgical tool or instrument during use. As one example, a tool drive shank or end of a surgical tool or instrument (not shown), such as, for example, a drill bit or screwdriver, is removably grasped or coupled within longitudinal bore 232 formed in tool or instrument engagement body 220. The drive shank of the surgical tool or instrument may be inserted into longitudinal bore 232 until the end of the drive shank or an end of the tool drive shank contacts a stop 234, at which point further insertion may be prevented. In one example, as the tool drive shank is being fully inserted into longitudinal bore 232, a living hinge resiliently attached to body 220 is adapted to cooperate with a corresponding groove or indentation on the outer surface of the tool drive shank inserted into longitudinal bore 232 of the tool or instrument engagement body 220. In this example, the living hinge may be accessible through a transverse opening formed in the side surface of tool or instrument engagement body for manual engagement by a user. In alternative embodiments, tool or instrument engagement body 220 may include a chuck to removably grasp the tool drive shank. In other embodiments, tool or instrument engagement body 220 may removably couple to the tool drive shank by an AO, square drive or Hudson style orthopedic instrument connection known in the art.

Mounting post 240 comprises, at distal end 214, a pedestal 250 and a cap 260. Pedestal 250 and cap 260 are configured for acceptance into a through-hole 432 formed in retainer base 430 of rear power housing 400, as illustrated in FIG. 2C. Pedestal 250 is attached to and extends axially along longitudinal axis 210 from mounting post 240. Pedestal 250 can have a variety of transverse heights depending upon the particular application and the particular dimensions of retainer base 430. The illustrated pedestal 250 has a generally cylindrical shape, be can be configured in a variety of other shape, which can match the shape of through-hole 432 formed in retainer base 430 of rear power housing 400.

Cap 260 extends radially outward from the top portion of pedestal 250. Cap 260 assists in coupling mounting post 240 of tool connector 200 to retainer base 430 of rear power housing 400 by inhibiting separation of pedestal 250 from retainer base 430. The illustrated cap 260 has a cross-sectional shape generally similar to that of pedestal 250 for ease of manufacture, however, it can be configured in a variety of other cross sectional shapes to generally match the shape of through-hole 432 in retainer base 430, which is described below. Cap 260 desirably extends beyond the circumference of pedestal 250 by a lip 268 to assist in securely coupling mounting post 240 to retainer base 430. Cap 260 need not circumscribe the entire pedestal 250 and can comprise only a one or more radial member that extends radially outwardly from pedestal 250. The transverse thickness of cap 260 may be sufficient to perform its structural function of coupling mounting post 240 to retainer base 430 without significantly bending or breaking.

A chamfer 262 may be formed on an upper peripheral edge of cap 260 to assist in the assembly of mounting post 240, as described below. In one example, the illustrated chamfer 262 transversely extends about one-half the thickness of cap 260. In one embodiment, pedestal 250 and cap 260 further include a hole or slot 264 extending axially through at least a portion of cap 260 and pedestal 250. Hole or slot 264 facilitates coupling between mounting post 240 and retainer base 430 via through-hole 432 in retainer base 430 by allowing portions of cap 260 to flex radially inwards as cap 260 is urged through the through-hole 432 during assembly, as described below.

Pedestal 250 desirably has a smooth side surface 252 to facilitate sliding and rotation of pedestal 250 relative to retainer base 430, such that pedestal 250 provides a bearing surface for retainer base 430. Lip 268 of cap 260 may include a flat underside surface 266 to match the configuration of a contacting surface 438 of retainer base 430 past through-hole 432 to provide a flush surface and a bearing surface for rotation of tool connector 200 relative to rear power housing 400, as described below. In the illustrated example, pedestal 250 and cap 260 have a one-piece configuration for ease of manufacture and strength. However, pedestal 250 and cap 260 can alternatively comprise a two-piece configuration extending from or attached to mounting post 240. Although the combination of pedestal 250 and cap 260 is generally mushroom shaped, pedestal 250 and cap 260 can also be generally T-shaped, inversely L-shaped and the like.

Pedestal 250 and cap 260 are desirably formed in unity with mounting post 240 for structural strength. However, pedestal 250 and cap 260 can comprise separate components. The illustrated pedestal 250, cap 260 and through-hole 432 of retainer base 430 have a circular configuration, with the longitudinal axis of both pedestal 250, cap 260 and through-hole of retainer base 430 being aligned with longitudinal axis of handle 1100 so that tool connector 200 can centrally rotate relative to rear power housing 400.

In the illustrated embodiment, as best shown in FIG. 2C, retainer base 430 of rear power housing 400 has a through-hole 432 sized and configured to receive pedestal 250 and more preferably to generally match that of pedestal 250 so that tool connector 200 can rotate relative to rear power housing 400 about pedestal 250. The illustrated through-hole 432 extends through retainer base 430 and has a first diameter. Through-hole 432 communicates with retainer space 436 that may have a second diameter. In one example, first diameter is slightly larger than that of pedestal 250 and second diameter of retainer space 436 is slightly larger than that of cap 260. Like pedestal 250, through-hole 432 has a smooth surface to minimize friction when tool connector 200 is rotated. In one embodiment, a chamfer (not shown) may circumscribe the lower portion of first diameter of through-hole 432 to assist in the assembly of the rotatable mounting post 240 of tool connector 200, as described below.

When assembled, pedestal 250 and cap 260 are inserted and transversely advanced into through-hole 432 and secured to retainer base 430. In particular, cap 260 is housed within retainer space 436 of retainer base 430 with the underside surface 266 of lip 268 of cap 260 being generally flush with contacting surface 438 in retainer space 436. Chamfer 262 that circumscribes cap 260 allows cap 260 to deform and advance through through-hole 432, aided, in some embodiments with, for example, a chamfer (not shown) that circumscribes the entrance of through-hole 432 from cavity 440. Once cap 260 passes through through-hole 432, cap 260 radially displaces and bounces back to its original configuration and underside surface 266 of lip 268 meshes with contacting surface 438 in retainer space 436, while pedestal 250 extends through through-hole 432. By this configuration, tool connector 200 can rotate three hundred and sixty degrees relative to rear power housing 400.

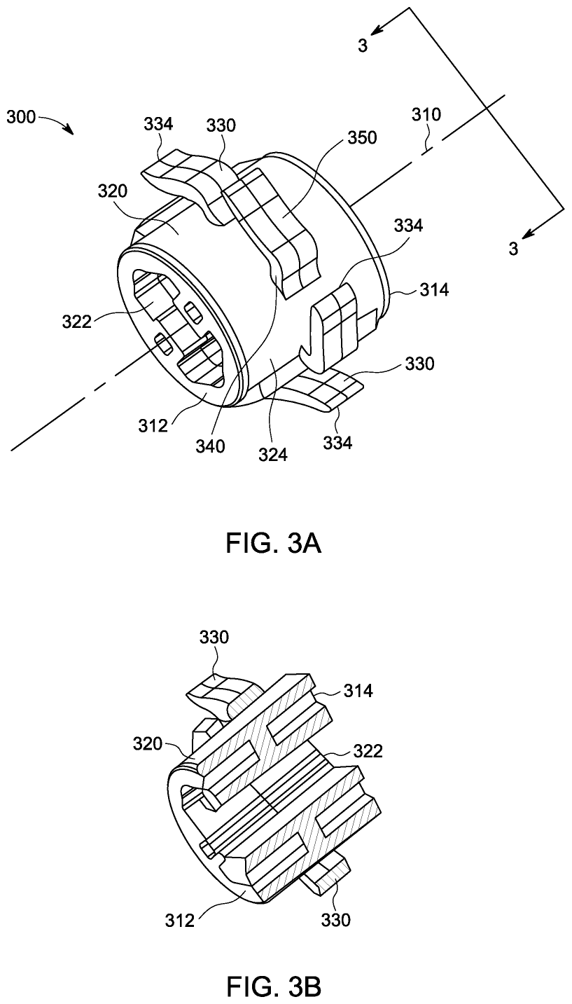

FIGS. 3A and 3B illustrate one example of a torque coupler 300 constructed in accordance with one or more aspects of the present invention. Torque coupler 300 includes a longitudinal axis 310, a proximal end 312 and a distal end 314. During operation, longitudinal axis 310 aligns with longitudinal axis 1100 of handle 1000. Torque coupler 300 also comprises a generally cylindrical body 320 having an outer surface 324 and defining a longitudinal through-hole 322. Longitudinal through-hole 322 is configured and shaped to slidably receive mounting post 240 of tool connector 200 during assembly. Through-hole 322 is also configured and shaped so that torque coupler 300 will rotate simultaneously with tool connector 200 during operation of handle 1000.

As illustrated in FIG. 3A, a plurality of hinges or fingers 330 project radially outward from outer surface 324. In one embodiment, as shown in FIG. 3A, there may be six fingers or hinges 330 spaced radially equidistant from around outer surface 324. Each hinge or finger 330 includes a proximal end 332, a distal end 334, a proximal portion 340 directly affixed to outer surface 324 at proximal end 332 and extending radially outward from outer surface 324 and a distal portion 350 extending from proximal portion 340 towards distal end 334. Proximal portion 340 may include a flare at the bottom to form an annular fillet 342. Annular fillet 342 provides structural strength to finger or hinge to resist shear and other forces that can otherwise cause finger or hinge 330 to break off from outer surface 324 of body 320 or otherwise fail. Distal portion 350 may bend at an angle relative to proximal portion 340 and further extend circumferentially around or followed around the circumference of a portion of body 324. Distal portion 350 may also include a radially outward facing surface 352.

In one embodiment, plurality of fingers or hinges 330 may be spaced radially equidistant from one another to allow the engagement between each finger or hinge 330 and teeth 450 on interior surface 442 of cavity 440 of body 420 of rear power housing 400. Each finger or hinge 330 is resilient, flexible and biased radially outward from longitudinal axis 310 of torque coupler 300. In one example, fingers or hinges 330 are integral with body 320 and formed during the same injection molding process. In alternative embodiments, fingers or hinges 330 may be created by additive manufacturing or may be metallic members that are assembled or molded by, for example, insert molding, to outer surface 324 of torque coupler 300.

A handle 1000 constructed in accordance with one or more aspects of the present invention is intended to limit the torque applied to protect instruments from torsional overload. While the illustrated embodiment may have six fingers or hinge 330 equidistantly spaced from one another around outer surface 324, a plurality of offset distances may be used as well to achieve substantially the same result or a different desired result. Further, the number of fingers//hinges 330 and/or the thickness and width of each finger or hinge 330 may be “tuned” or vary greatly depending on the particular load or force desired for a particular application (e.g. desired torque for fingers or hinges 330 to overcome or pass over teeth 450 of rear power housing 400 in a particular direction) by each finger or hinge 330. The particular number, configuration and design of the plurality of fingers or hinges 330 can be varied to accommodate the various loads or forces that may be needed or desired therethrough during operation of handle 1000 and, for example, convey to a surgeon or user that a desired torque has been achieved. For example, the fingers or hinges 330 illustrated in FIG. 3A are configured and design to require approximately 1 N/m to overcome or pass over teeth 450 of rear power housing 400 when using a 2.5 mm screw so as not to break the screw.

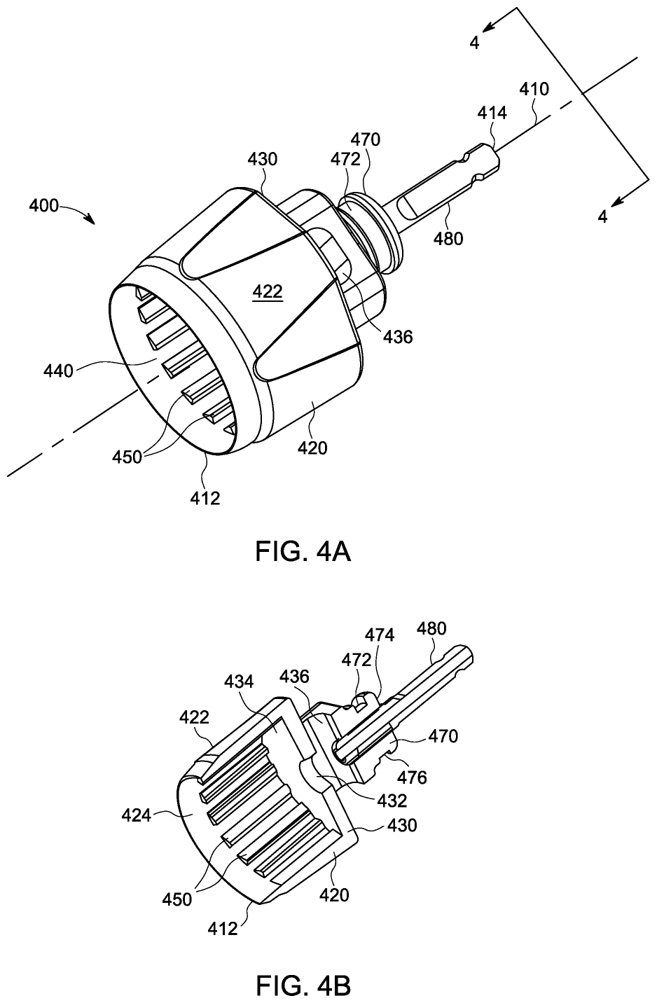

FIGS. 4A and 4B illustrate one example of a rear power housing 400 constructed in accordance with one or more aspects of the present invention. Rear power housing 400 includes a longitudinal axis 410, a proximal end 412 and a distal end 414. During operation, longitudinal axis 410 aligns with longitudinal axis 1100 of handle 1000 during assembly and use. As illustrated in FIG. 4A, rear power housing 400 comprises body 420 extending from proximal end 41, a drive shank base 470 extending from body 420, and a drive shank 480 coupled to and extending from drive shank base 470 longitudinally along longitudinal axis 410 to distal end 414. Drive shank base 470 permanently couples or holds drive shank 480.

As illustrated in FIG. 4A, body 420 is generally cylindrical in shape that may include a cylindrical side wall 422 and a retainer base 430 that together define a longitudinal cavity 440 open at proximal end 412. Cavity 440 communicates with a retainer space 436 via through-hole 432, described above. Cavity 440 is defined by interior surface 424 of side walls 422 and inner surface 434 of retainer base 430. Retainer space 436 is defined by contacting surface 438 and drive shank base 470.

As shown in FIGS. 4A and 4B, interior surface 424 of side wall 422 includes a plurality of teeth 450 projecting radially inward towards longitudinal axis 410. Plurality of teeth 450 are spaced radially equidistant or disposed at intervals in the circumferential direction around and extend axially on interior surface 424 along longitudinal axis 410. As illustrated clearly in FIG. 8C, each tooth 450 includes an inclined surface 452 and a stop surface 454. Inclined surface 452 angles radially inward to create a ramp. Stop surface 454 extends radially outward from interior surface 424 and substantially transverse to longitudinal axis 410. A slot or space 460 may be formed by interior surface 424 between a stop surface 454 of one tooth 450 and an inclined surface 452 of an adjacent tooth 450.

The particular number, configuration and design of the plurality of teeth 450 may be varied to accommodate the various loads or forces that may be needed or desired therethrough during operation of handle 1000. Further, the number of teeth 450 and/or the height and length of inclined surface 452 of each tooth 450 may be “tuned” or vary greatly depending on the particular load or force desired for a particular application (e.g. desired torque for fingers or hinges 330 to overcome or pass over teeth 450 of rear power housing 400 in a particular direction) by each tooth 450.

Drive shank 480 may be configured and designed to couple to various types of power instruments to drive handle 1000. For example, as illustrated in FIG. 4A, drive shank 480 comprises be a hex drive shank that includes a quick connect feature. A hex drive shank design provides for high torque transmission and have no need to be tightened. A hex drive shank design also does not allow for slipping commonly experienced with straight cylindrical drive shanks. In alternative embodiments, drive shank may be in the form of other known drive shank shapes, such as, for example, SDS drive shanks, straight drive shanks, square drive shank, triangle drive shanks or the like. Drive shank 480 may also be designed to be, for example, removably coupled to power instruments comprising one of an AO, square drive, or Hudson® style orthopedic instrument connection.

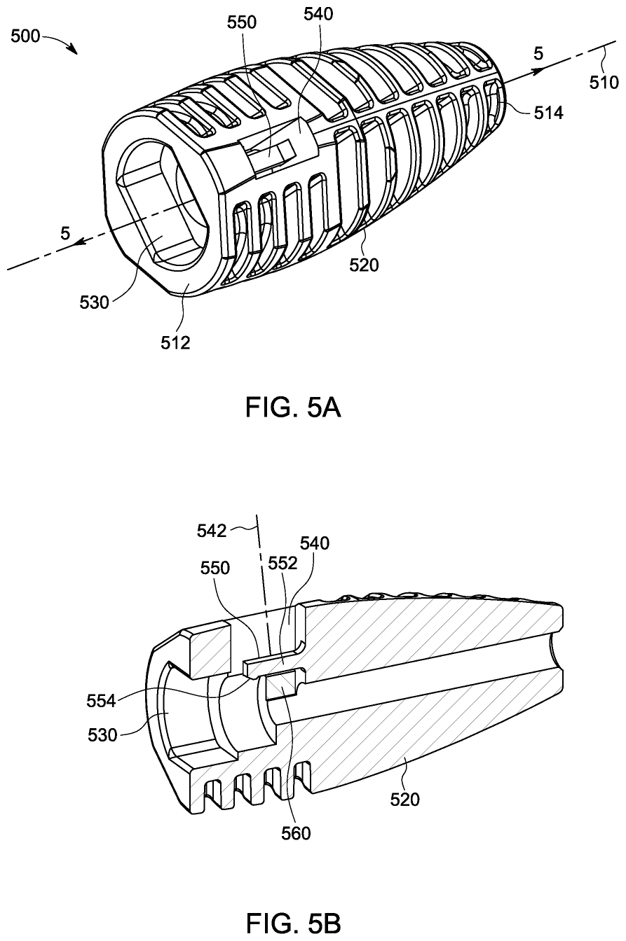

Drive shank 480 may also be removably coupled to an optional handle grip constructed in accordance with one or more aspects of the present invention. FIGS. 5 and 5A illustrate a perspective and cross-sectional view, respectively, of one example of an optional handle grip 500 constructed in accordance with one or more aspects of the present invention. As illustrated in FIG. 5A, handle grip 500 may include a body 520 having a longitudinal axis 510, a proximal end 512 and a distal end 514. During assembly and operation, longitudinal axis 510 aligns with longitudinal axis 1100 of handle 1000.

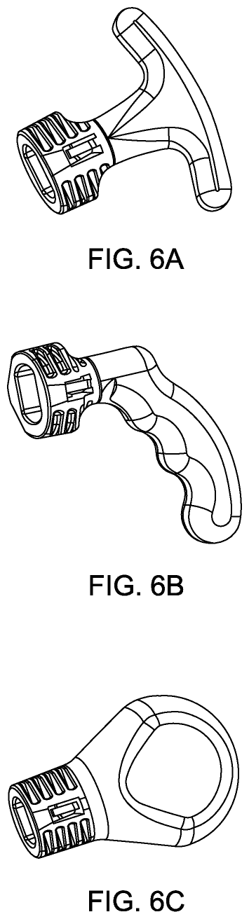

One example, as illustrated in FIG. 5A, of body 520 may have a bulbous shape suitable for being held by a human hand. Other examples of body-shapes for handle grip 500 are illustrated in FIGS. 6A-6C, which include, for example, a T-handle configuration (FIG. 6A), a pistol grip (FIG. 6B) or a palm handle (FIG. 6C). Handle grip 500 may also be in the form of, for example, a ball or any other various shaped configurations that permit a user to manually apply torque to the surgical instrument or tool attached to handle 1000. In other embodiments, handle grip 500 may be customizable in applications for various commercial marketing purposes with respect to, for example, color, marking and texture.

Body 520 of handle grip 500 may have a light weight, inexpensive, biologically inert material. In one example, handle grip 500 may be made from polyacrylamide, polycarbonate or acrylonitrile butadiene styrene (“ABS”). Handle grip 500 may also be a uni-body or monolithic design as shown in FIG. 5A. This uni-body construction makes handle grip 500 easier to manufacture and stronger than a multicomponent design having the same materials of construction.

Handle grip 500 may include a longitudinal bore 530 disposed through handle grip 500 along longitudinal axis 510. Longitudinal bore 530 is open at proximal end 512 of handle grip 500.

Handle grip 500 may also include a transverse bore 540. Transverse bore 540 is disposed through body 520 of handle grip 500. Transverse bore 540 may have a longitudinal axis 542. Transverse bore 540 intersects with longitudinal bore 530. In one example, longitudinal axis 542 of transverse bore 540 is perpendicular to longitudinal axis 510 of longitudinal bore 530. Transverse bore 540 may also have a first opening that opens out of body 520 and a second opening communicating with longitudinal bore 530.

Handle grip 500 further may include a button 550. In one embodiment, button 550 is flexibly attached to handle grip 500 as shown in FIGS. 5A and 5B. Button 550 may extend through transverse bore 540, intersecting longitudinal bore 530. Button 550 and transverse bore 540 may be disposed on body 520 of handle grip 500 such that button 550 is thumb accessible and/or depressible. Positioning button 550 closer to proximal end 512 of handle grip 500, also positions button 550 closer to the portion of handle grip 500 that engages with a groove 472 formed in drive shank housing 470 of rear power housing 400. Handle grip 500 may be configured (e.g. shaped and dimensions) to allow handle grip 500 to be held, grasped, or used by a hand such that the fifth digit and hypothenar region are positioned in proximity to or around the distal end 514 of handle grip 500, with handle grip 500 extending across the palm and in the direction of the region between the first and second digit, such that the first digit or thumb may easily access and depress button 500.

Advantageously, because of the uni-body design, devices made in accordance with the present invention may not have additional components such as springs. Button 550 is connected to body by resilient member 552. Thus, a handle grip 500 constructed in accordance with one or more aspects of the present invention may be less expensive to manufacture and simple to use. Moreover, because the handle grip 500 is inexpensive to make, it is an ideally suited single use (e.g. disposable) device. Cleanliness is assured because the handle grip 500 is removed from a sterile package and used only once.

Referring now to FIG. 5B, there is shown a cross-sectional view of a handle grip 500 constructed in accordance with one or more aspects of the present invention. As illustrated, handle grip 500 includes a backstop 560 disposed within longitudinal bore 530. Button 550 may also have a thickness which may extend into transverse bore 540 in a longitudinal direction relative to longitudinal axis 542. In one embodiment, button 550 includes a distal end having a lip 554. Lip 554 projects and is normally biased radially inward towards longitudinal axis 510 of handle grip 500. In one embodiment, button 550 with lip 554 creates a living hinge when coupled to rear power housing 400.

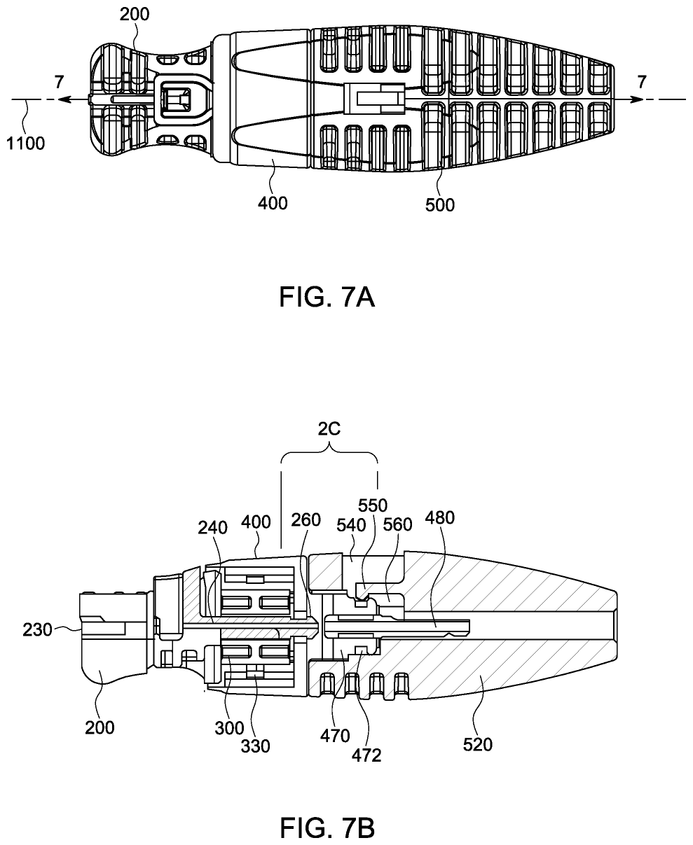

Referring to FIG. 7A, drive shank 480 of rear power housing 400 can be inserted through proximal end 512 into longitudinal bore 530 of handle grip 500. Drive shank 480 of rear power housing 400 may be inserted into longitudinal bore 530 until, for example, end surface 474 of drive shank base 470 of rear power housing 400 contacts boss 560 or, in another example, until contacting surface 438 of retainer base 430 of rear power housing 400 contacts proximal end 512 of handle grip 500, at which point further insertion may be inhibited. As drive shank 480 is being fully inserted into longitudinal bore 530, lip 554 of button 550 slides into groove 472 formed on the outer surface 476 of drive shank base 470 of rear power housing 400. Lip 554 of button 550 is adapted to cooperate with corresponding groove or indentation 472 on outer surface 476 of drive shank base 470 of rear power housing 400 inserted into longitudinal bore 530 of handle grip 500.

Once drive shank 480 and drive shank base 470 of rear power housing 400 are inserted into handle grip 500, lip 554 of button 550 is biased into groove 472. In one embodiment, a “clicking” sound may be heard when lip 554 fully engages groove 472. However, a user may disengage rear power housing 400 from handle grip 500 by forcibly pulling out rear power housing 400 from handle grip 500 such that lip 554 pivots out of groove 472 within drive shank base 470 of rear power housing 400. Rear power housing 400 may connect with lip 554 fitted into groove 472 providing significant resistance to disengagement forces. However, rear power housing 400 may still be pulled in response to significant force being applied by a user to rear power housing 400 through handle grip 500. In one example, a transverse force may be applied to drive shank base 470 of rear power housing 400 by depressing button 550, providing additional force to prevent rear power housing 400 from being pulled out by disengagement forces.

In other embodiments, handle grip 500 may include more than one buttons or living hinges 330 engaged with groove 472 formed in drive shank base 470 of rear power housing 400. Alternatively, other coupling mechanisms may be applied to drive shank 480 or drive shank base 470 of rear power housing 400 to removably retain within handle grip 500 during use. For example, the coupling mechanisms described and illustrated in WO2019/168987, which is hereby incorporated herein by reference, may be used. In other example, drive shank 480 may also be designed to be, for example, removably coupled to handle grip 500 comprising one of an AO, square drive, or Hudson® style orthopedic instrument connection.

In one embodiment, rotation can be applied to rear power housing 400 either directly to drive shaft 480 by, for example, a power instrument, or directly to other aspects of rear power housing 400 (e.g. drive shank base 470) by, for example, manual rotation to handle grip 500.

When assembled, torque coupler 300 is slide over mounting post 240 of tool connector 200. Then, distal end 414 of tool connector 200 is inserted axially into cavity 440 formed in body 420 of rear power housing 400. Pedestal 250 and cap 260 of tool connector 200 are inserted and transversely advanced into through-hole 432 formed in retainer base 430 of rear power housing 400. Cap 260 is advanced completely through through-hole 432 until cap 260 is housed completely within retainer space 436 with underside surface 266 of lip 268 of cap 260 being generally flush with contacting surface 438 of retainer base 430 in retainer space 436. Once cap 260 is fully seated within retainer space 436, distal connecter 200 with torque coupler 300 are able to rotate, but not move in an axial direction, relative to rear power housing 400. And, plurality of hinges or fingers 330 of torque coupler 300 engage plurality of teeth 450 of rear power housing 400. At this point, tool connector 200, torque coupler 300 and rear power housing 400 are assembled together for use with either optional handle grip 500 or a power instrument attachable to drive shaft 480 of rear power housing 400.

In operation, handle 1000 may be used to, for example, screw a fastener into bone during, for example, an orthopedic extremity, large joint or spinal surgery. First, a drive shaft of a screw or drill bit may be inserted through tool or instrument opening 230 into longitudinal bore 232 of tool connector 200 and removably coupled within by a coupling mechanism. If the surgeon or user desires to manually insert the screw, handle grip 500 is removably coupled to drive shank 480 of rear power housing 400 by, for example, inserting distal end 414 of drive shank 480 into longitudinal bore 530 at proximal end 512 of handle grip 500 until lip 554 of button 550 engages groove 472 of shank base 470 of rear power housing 400 or unless stopped by, for example, boss 560 or proximal end 512 of handle grip 512. If the surgeon or user desires to insert the screw using, for example, a power drill or instrument, drive shank 480 is removably attached to the coupling mechanism of the power drill or instrument. In accordance with one or more aspects of the present invention, handle 1000 is designed for a surgeon or user to easily transition between power and manual application of torque.

While inserting the screw into bone using manual power, the surgeon or user would grab handle grip 500 with one hand and apply a clockwise rotational motion to handle grip 500. Rear power housing 400 also simultaneously rotates clockwise with handle grip 500. Referring to FIG. 8C, as rear power housing 400 is rotated clockwise for the purpose of inserting a screw, each of the plurality of fingers of hinges 330 of torque coupler 300 will flex radially inward as their radially outward facing surfaces 352 of distal portions 350 slidably engage and pass or break over inclined surfaces 452 of teeth 450, imparting rotation in the clockwise direction. As handle grip 500/rear power housing 400 continue to rotate clockwise, each of the plurality of fingers 330 will slide over apex 456 of each tooth 330 and bounce back or flex radially outward (to their original shape) and into slot or space 460 between adjacent teeth 450. In some embodiments, the surgeon or user will hear a clicking should as fingers 330 pass over a tooth 450 into slot or space 460. Continuing clockwise rotation of handle grip 500/rear power housing 400 will result in the plurality of fingers 330 slidably engaging inclined surface 452 of an adjacent tooth 450 and pass over into the next slot or space 460. The rotation of tool connector 200, torque coupler 300, the screw bit and the screw will be dictated (e.g. limited) by the amount of torque allowed by the interaction between the fingers or hinges 330 of torque coupler 300 and the teeth 450 with rear power housing 400.

The interaction of the plurality of fingers or hinges 330 and the plurality of teeth 450 in operation govern or limit the torque being applied by the surgeon or user. In other words, the interaction of the fingers or hinges 330 and teeth 450 limit the amount of torque that can be applied. This limiting of torque applied is intended to protect, for example, the screw and/or bone from torsional overload. The torque being applied may also, for example, convey to the surgeon or user that a desired torque has been achieved. This desired torque, as discussed above, may be set or tuned by the particular number, configuration and design of the plurality of fingers/hinges 330 and/or teeth 450.

When handle 1000 is used to, for example, remove a screw from bone, a surgeon or user would apply a counter-clockwise rotational motion to handle grip 500. Rear power housing 400 also simultaneously rotates counter-clockwise with handle grip 500. As rear power housing 400 is rotated counter-clockwise for the purpose of removing a screw, distal end 334 of each of the plurality of fingers of hinges 330 of torque coupler 300 will wedge or lock into stop surfaces 454 of teeth 450 to prevent further rotation of torque coupler 300 (as illustrated by the configuration shown in FIG. 8C). Once distal end 334 of each of the plurality of fingers or hinges 330 are wedged or locked into place at stop surface 454 of teeth 450, plurality of fingers or hinges 330 will not be able to flex or freely pass over the teeth 450 to permit break over. In this configuration, maximal torsional may be applied by a surgeon or user to remove a screw.

Tool connector 200, torque coupler 300, rear power housing 400 and optional handle grip 500 may all be manufactured by, for example injection molding, additive manufacturing or 3D printing. Also, each of these components may be cannulated along the longitudinal axis to permit passage of, for example, guidewires or K-wire, therethrough.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. An apparatus for releasably holding a surgical tool, said apparatus comprising:
a tool connector, said tool connector including a longitudinal axis, a proximal end and a distal end, said tool connector further including a tool engagement body and a mounting post extending longitudinally along the longitudinal axis from the tool engagement body to the distal end, the tool engagement body including a tool engagement opening at the proximal end communicating with a longitudinal bore extending through at least a portion of the tool engagement body along the longitudinal axis, the longitudinal bore configured to releasably coupled the surgical tool;
a torque coupler, said torque coupler including a cylindrical body defining a through hole and an outer surface, the mounting post of said tool connector passing through the through hole of the cylindrical body, said torque coupler further including a plurality of fingers extending radially outward from the outer surface;
a rear power housing, said rear power housing rotatably coupled to the mounting post of said tool connector at the distal end, said rear power housing including a longitudinal axis, a body and a drive shaft extending longitudinally along the longitudinal axis from the body of said rear power housing, the body including a cavity defining an inner surface and a plurality of teeth projecting radially inward from the inner surface, wherein, during rotation of said rear power housing in a first direction, the plurality of fingers slidably engage the plurality of teeth to limit the applied torque of said torque coupler and said tool connector from said rear power housing, and wherein, during rotation of said rear power housing in a second direction, the plurality of teeth prevent movement of the plurality of fingers to allow maximal applied torque of said torque coupler and said tool connector from said rear power housing.

2. The apparatus for releasably holding a surgical tool of claim 1, wherein said rear power housing is removeably attachable to a handle grip.

3. The apparatus of claim 2, wherein said handle grip comprises a body, the body including a longitudinal axis and a longitudinal bore disposed along the longitudinal axis of the body, the longitudinal bore open at one end and configured to receive and removeably couple said rear power housing.

4. The apparatus of claim 3, wherein said rear power housing is removeably coupled within the longitudinal bore by a living hinge.

5. The apparatus of claim 2, wherein said handle grip comprises a body, the body in a shape apportioned to be grasped by a human hand.

6. The apparatus of claim 2, wherein said handle grip comprises a body, at least a portion of the body in a shape of a T-handle.

7. The apparatus of claim 2, wherein said handle grip comprises a body, at least a portion of the body in a shape of a pistol grip.

8. The apparatus of claim 2, wherein said handle grip comprises a body, at least a portion of the body in a shape of a palm handle.

9. The apparatus of claim 2, wherein said handle grip comprises a body, at least a portion of the body in a shape of a ball.

10. The apparatus of claim 1, wherein the drive shaft is removeably attachable to a power instrument.
